SEQUENCE LISTING (31 sequences)

SEQ ID NO: 1 - skipped

SEQ ID NO: 2 - DNA, 19 bp, synthetic construct
  source 1..19: mol_type = other DNA
ataattgctgatgtaatgt

SEQ ID NO: 3 - DNA, 19 bp, synthetic construct
  source 1..19: mol_type = other DNA
atatttgctgaattaaatc

SEQ ID NO: 4 - DNA, 19 bp, synthetic construct
  source 1..19: mol_type = other DNA
aaatgtgctgactcagaga

SEQ ID NO: 5 - DNA, 19 bp, synthetic construct
  source 1..19: mol_type = other DNA
aatagtgctgatgctgtgt

SEQ ID NO: 6 - skipped

SEQ ID NO: 7 - DNA, 17 bp, synthetic construct
  source 1..17: mol_type = other DNA
aggtcannnngtgacct

SEQ ID NO: 8 - DNA, 16 bp, synthetic construct
  source 1..16: mol_type = other DNA
tgacctttaaaggtca

SEQ ID NO: 9 - DNA, 16 bp, synthetic construct
  source 1..16: mol_type = other DNA
tgacctttaaagatca

SEQ ID NO: 10 - DNA, 11 bp, synthetic construct
  source 1..11: mol_type = other DNA
tctaaaggtca

SEQ ID NO: 11 - DNA, 17 bp, synthetic construct
  source 1..17: mol_type = other DNA
aggtcaacttgtgacct

SEQ ID NO: 12 - DNA, 210 bp, synthetic construct
  source 1..210: mol_type = other DNA
gatctttgttttgattaaataattgctgatgtaatgtcgcgatgtacgtaagataattgctgatgtaatgttgctgataagataattgctgatgtaatgtagacgtggcagttagataattgctgatgtaatgtggggattaagataattgctgatgtaatgtgcgattaagctttccaatatgctctagagggtatataatgggggcca

SEQ ID NO: 13 - DNA, 244 bp, synthetic construct
  source 1..244: mol_type = other DNA
gatctttgttttgattaaataattgctgatgtaatgtcgcgatgtacgtaagataattgctgatgtaatgttgctgataagataattgctgatgtaatgtagacgtggcagtaagataattgctgatgtaatgtggggattaagataattgctgatgtaatgtgcgattaagctttccaatatgcgtaggcgtgtacggtgggaggtctatataagcagagctcgtttagtgaaccgtcagatc

SEQ ID NO: 14 - DNA, 248 bp, synthetic construct
  source 1..248: mol_type = other DNA
gatctttgttttgattaaataattgctgatgtaatgtcgcgatgtacgtaagataattgctgatgtaatgttgctgataagataattgctgatgtaatgtagacgtggcagtaagataattgctgatgtaatgtggggattaagataattgctgatgtaatgtgcgattaagctttccaatatgcttcgcatattaaggtgacgcgtgtggcctcgaacaccgagcgaccctgcagcgacccgcttaa

SEQ ID NO: 15 - DNA, 236 bp, synthetic construct
  source 1..236: mol_type = other DNA
gatctttgttttgattaaataattgctgatgtaatgtcgcgatgtacgtaagataattgctgatgtaatgttgctgataagataattgctgatgtaatgtagacgtggcagtaagataattgctgatgtaatgtggggattaagataattgctgatgtaatgtgcgattaagctttccaatatgcagacgctagcggggggctataaaagggggtgggggcgttcgtcctcactct

SEQ ID NO: 16 - DNA, 210 bp, synthetic construct
  source 1..210: mol_type = other DNA
gatctttgttttgattaaatatttgctgaattaaatccgcgatgtacgtaagatatttgctgaattaaatctgctgataagatatttgctgaattaaatcagacgtggcagttagatatttgctgaattaaatcggggattaagatatttgctgaattaaatcgcgattaagctttccaatatgctctagagggtatataatgggggcca

SEQ ID NO: 17 - DNA, 244 bp, synthetic construct
  source 1..244: mol_type = other DNA
gatctttgttttgattaaatatttgctgaattaaatccgcgatgtacgtaagatatttgctgaattaaatctgctgataagatatttgctgaattaaatcagacgtggcagtaagatatttgctgaattaaatcggggattaagatatttgctgaattaaatcgcgattaagctttccaatatgcgtaggcgtgtacggtgggaggtctatataagcagagctcgtttagtgaaccgtcagatc

SEQ ID NO: 18 - DNA, 248 bp, synthetic construct
  source 1..248: mol_type = other DNA
gatctttgttttgattaaatatttgctgaattaaatccgcgatgtacgtaagatatttgctgaattaaatctgctgataagatatttgctgaattaaatcagacgtggcagtaagatatttgctgaattaaatcggggattaagatatttgctgaattaaatcgcgattaagctttccaatatgcttcgcatattaaggtgacgcgtgtggcctcgaacaccgagcgaccctgcagcgacccgcttaa

SEQ ID NO: 19 - DNA, 236 bp, synthetic construct
  source 1..236: mol_type = other DNA
gatctttgttttgattaaatatttgctgaattaaatccgcgatgtacgtaagatatttgctgaattaaatctgctgataagatatttgctgaattaaatcagacgtggcagtaagatatttgctgaattaaatcggggattaagatatttgctgaattaaatcgcgattaagctttccaatatgcagacgctagcggggggctataaaagggggtgggggcgttcgtcctcactct

SEQ ID NO: 20 - DNA, 210 bp, synthetic construct
  source 1..210: mol_type = other DNA
gatctttgttttgattaaaaatgtgctgactcagagacgcgatgtacgtaagaaatgtgctgactcagagatgctgataagaaatgtgctgactcagagaagacgtggcagttagaaatgtgctgactcagagaggggattaagaaatgtgctgactcagagagcgattaagctttccaatatgctctagagggtatataatgggggcca

SEQ ID NO: 21 - DNA, 244 bp, synthetic construct
  source 1..244: mol_type = other DNA
gatctttgttttgattaaaaatgtgctgactcagagacgcgatgtacgtaagaaatgtgctgactcagagatgctgataagaaatgtgctgactcagagaagacgtggcagtaagaaatgtgctgactcagagaggggattaagaaatgtgctgactcagagagcgattaagctttccaatatgcgtaggcgtgtacggtgggaggtctatataagcagagctcgtttagtgaaccgtcagatc

SEQ ID NO: 22 - DNA, 248 bp, synthetic construct
  source 1..248: mol_type = other DNA
gatctttgttttgattaaaaatgtgctgactcagagacgcgatgtacgtaagaaatgtgctgactcagagatgctgataagaaatgtgctgactcagagaagacgtggcagtaagaaatgtgctgactcagagaggggattaagaaatgtgctgactcagagagcgattaagctttccaatatgcttcgcatattaaggtgacgcgtgtggcctcgaacaccgagcgaccctgcagcgacccgcttaa

SEQ ID NO: 23 - DNA, 236 bp, synthetic construct
  source 1..236: mol_type = other DNA
gatctttgttttgattaaaaatgtgctgactcagagacgcgatgtacgtaagaaatgtgctgactcagagatgctgataagaaatgtgctgactcagagaagacgtggcagttagaaatgtgctgactcagagaggggattaagaaatgtgctgactcagagagcgattaagctttccaatatgcagacgctagcggggggctataaaagggggtgggggcgttcgtcctcactct

SEQ ID NO: 24 - DNA, 209 bp, synthetic construct
  source 1..209: mol_type = other DNA
gatctttgttttgattaaaatagtgctgatgctgtgtcgcgatgtacgtaagaatagtgctgatgctgtgttgctgataagaatagtgctgatgctgtgtagacgtggcagtaagaatagtgctgatgctgtgtggggattaagaatagtgctgatgctgtgtgcgattaagcttccaatatgctctagagggtatataatgggggcca

SEQ ID NO: 25 - DNA, 244 bp, synthetic construct
  source 1..244: mol_type = other DNA
gatctttgttttgattaaaatagtgctgatgctgtgtcgcgatgtacgtaagaatagtgctgatgctgtgttgctgataagaatagtgctgatgctgtgtagacgtggcagtaagaatagtgctgatgctgtgtggggattaagaatagtgctgatgctgtgtgcgattaagctttccaatatgcgtaggcgtgtacggtgggaggtctatataagcagagctcgtttagtgaaccgtcagatc

SEQ ID NO: 26 - DNA, 248 bp, synthetic construct
  source 1..248: mol_type = other DNA
gatctttgttttgattaaaatagtgctgatgctgtgtcgcgatgtacgtaagaatagtgctgatgctgtgttgctgataagaatagtgctgatgctgtgtagacgtggcagtaagaatagtgctgatgctgtgtggggattaagaatagtgctgatgctgtgtgcgattaagctttccaatatgcttcgcatattaaggtgacgcgtgtggcctcgaacaccgagcgaccctgcagcgacccgcttaa

SEQ ID NO: 27 - DNA, 236 bp, synthetic construct
  source 1..236: mol_type = other DNA
gatctttgttttgattaaaatagtgctgatgctgtgtcgcgatgtacgtaagaatagtgctgatgctgtgttgctgataagaatagtgctgatgctgtgtagacgtggcagtaagaatagtgctgatgctgtgtggggattaagaatagtgctgatgctgtgtgcgattaagctttccaatatgcagacgctagcggggggctataaaagggggtgggggcgttcgtcctcactct

SEQ ID NO: 28 - DNA, 195 bp, synthetic construct
  source 1..195: mol_type = other DNA
gatctttgttttgattaatgacctttaaaggtcacgcgatgtacgtaagtgacctttaaaggtcatgctgataagtgacctttaaaggtcaagacgtggcagtaagtgacctttaaaggtcaggggattaagtgacctttaaaggtcagcgattaagctttccaatatgctctagagggtatataatgggggcca

SEQ ID NO: 29 - DNA, 200 bp, synthetic construct
  source 1..200: mol_type = other DNA
gatctttgttttgattaaaggtcaacttgtgacctcgcgatgtacgtaagaggtcaacttgtgaccttgctgataagaggtcaacttgtgacctagacgtggcagtaagaggtcaacttgtgacctggggattaagaggtcaacttgtgacctgcgattaagctttccaatatgctctagagggtatataatgggggcca

SEQ ID NO: 30 - DNA, 170 bp, synthetic construct
  source 1..170: mol_type = other DNA
gatctttgttttgattaatctaaaggtcacgcgatgtacgtaagtctaaaggtcatgctgataagtctaaaggtcaagacgtggcagtaagtctaaaggtcaggggattaagtctaaaggtcagcgattaagctttccaatatgctctagagggtatataatgggggcca

SEQ ID NO: 31 - DNA, 195 bp, synthetic construct
  source 1..195: mol_type = other DNA
gatctttgttttgattaatgacctttaaagatcacgcgatgtacgtaagtgacctttaaagatcatgctgataagtgacctttaaagatcaagacgtggcagtaagtgacctttaaagatcaggggattaagtgacctttaaagatcagcgattaagctttccaatatgctctagagggtatataatgggggcca

(19) United States
(12) Patent Application Publication
(10) Pub. No.: US 2026/0357273 A1
(43) Pub. Date: Nov. 12, 2026

(54) Engineered Nucleic Acids And Uses Thereof As Sensors Of T-Cell Exhaustion

(71) Applicant: FONDAZIONE ISTITUTO ITALIANO DI TECNOLOGIA, Genova (IT)

(72) Inventors: Giuliano BONFA', Napoli (IT); Fabio CALIENDO, Boston, MA (US); Velia SICILIANO, Napoli (IT)

(21) Appl. No.: 19/485,725

(22) PCT Filed: May 23, 2024
(86) PCT No.: PCT/EP2024/064155

§ 371 Date: Nov. 19, 2025

(30) Foreign Application Priority Data
May 25, 2023 (IT) 102023000010581

(51) Int. Cl. C12N 15/63; A61K 38/19; A61K 38/20; A61K 48/00; A61P 35/00; C07K 14/52; C07K 14/54

(52) U.S. Cl. CPC C12N 15/635; A61K 38/195; A61K 38/208; A61K 48/005; A61P 35/00; C07K 14/523; C07K 14/5434

(57) ABSTRACT

The present invention relates to an engineered nucleic acid comprising a promoter and an immunomodulator, wherein the activity of said promoter is regulated by a protein involved in T cell exhaustion. The nucleic acids of the invention allow to prevent and/or reverse cell exhaustion T. Uses of engineered nucleic acid as a medicine, and in particular in the treatment of tumors and in immunotherapy, are described. Uses for inducing activity and sensing the exhaustion/dysfunctional state of a chimeric antigen receptor (CAR) T cell are also described.

### FIELD OF THE INVENTION

The present invention relates to an engineered nucleic acid comprising a promoter and an immunomodulator, wherein the activity of said promoter is regulated by a protein involved in T cell exhaustion. The nucleic acids of the invention allow to prevent and/or reverse T cell exhaustion.

Uses of the engineered nucleic acid as a medicament, and in particular in the treatment of tumors and in immunotherapy, are described.

Uses for inducing activity and sensing the exhaustion/dysfunctional state of a chimeric antigen receptor (CAR) T cell are also described.

### STATE OF THE ART

Advanced immunotherapy using adoptive transfer of engineered cells has become a clinical reality for the treatment of cancer patients since CAR T cell therapy was approved by the FDA. Despite this success, cell therapy still shows some limitations since, after chronic antigen stimulation, T cells become dysfunctional and are unable to suppress the tumor. T cell exhaustion is a process in which T cells progressively lose their functional properties and are therefore unable to react against the tumor. This process is characterized by loss of functionality (e.g. cytokine production) as well as transcriptional and epigenetic changes, with upregulation, among others, of key transcription factors (TFs).

Genetic devices capable of sensing the emergence of exhaustion have a fundamental dual application: 1) they can inform us about dynamic changes that T cells undergo during their anti-tumor activity. This would allow us to better design the timing of immunotherapy supporting CAR or endogenous T cells and provide insight into the molecular mechanisms underlying the dysfunction; 2) by coupling the sensors to transcriptional activation of therapeutic output we can reprogram T cells and restore their anti-tumor capacity.

During chronic infection and cancer, natural and engineered T cells (CAR) submitted to long-term antigen exposure become exhausted. This dysfunctional state compromises the optimal function of immune cells, which lose their cytotoxic and proliferative capacity. In the context of tumor treatment, T cell exhaustion significantly hinders therapy efficacy. A major limitation, so far, is the ability to rapidly recognize emerging dysfunction to promptly initiate immunotherapies. More importantly, designing T cells that can sense and counteract exhaustion would significantly improve CD8 T cell fitness and thus their efficacy.

One of the general objects of the present invention therefore consists in providing a product that finds application in the treatment of tumor states, through sensing and reversal of T cell exhaustion.

### SUMMARY OF THE INVENTION

The applicant of the present invention observed that a specific engineered nucleic acid comprising a promoter and an immunomodulator is endowed with properties capable of preventing and/or reversing T cell exhaustion and finds application in improving T cell activation and function and in cancer treatment.

The invention therefore concerns an engineered nucleic acid construct comprising:
a nucleic acid sequence encoding an inducible synthetic promoter, which is regulated by a protein involved in T cell exhaustion; and
a nucleic acid sequence encoding an immunomodulator under the control of said promoter, said immunomodulator being capable of preventing and/or reversing T cell exhaustion, and of enhancing T cell activation and function.

In a second aspect, the present invention concerns a virus comprising the engineered nucleic acid.

In a third aspect, the present invention concerns a cell comprising the engineered nucleic acid.

In a fourth aspect, the cell comprising the engineered nucleic acid according to the present invention or the virus comprising the engineered nucleic acid according to the present invention, for use as a medicinal product, is described.

In a fifth aspect, the cell comprising the engineered nucleic acid according to the present invention or the virus comprising the engineered nucleic acid according to the present invention, for use in the treatment of cancer, is described.

In a sixth aspect, the cell comprising the engineered nucleic acid according to the present invention or the virus comprising the engineered nucleic acid according to the present invention, for use in immunotherapy, is described.

In a seventh aspect, the cell comprising the engineered nucleic acid according to the present invention or the virus comprising the engineered nucleic acid according to the present invention, for inducing the activity of a chimeric antigen receptor (CAR) T cell, is described.

In an eighth aspect, the cell comprising the engineered nucleic acid according to the present invention or the virus comprising the engineered nucleic acid according to the present invention, for sensing the exhaustion/dysfunctional state of a chimeric antigen receptor (CAR) T cell, is described.

In a ninth aspect, the cell comprising the engineered nucleic acid according to the present invention or the virus comprising the engineered nucleic acid according to the present invention, for regulating gene expression in cell therapy by a chimeric antigen receptor (CAR) cell T, is described.

In a further aspect, a treatment method using the engineered nucleic acid according to the present invention, the cell comprising the engineered nucleic acid according to the present invention or the virus comprising the engineered nucleic acid according to the present invention, is described.

The dependent claims describe particular embodiments of the invention.

### DESCRIPTION OF THE FIGURES

The invention will now be described in detail and with reference to the attached Figures in which:

FIG. 1. Structure of the synthetic promoter and expected performance. A) Schematic view of the Synthetic Promoter (SP), made of responsive elements in which binding sites of the transcription factor (TF-BS) are cloned in 3-5× tandem repeats upstream of a minimal core promoter (YB_TATA, mini CMV, mini TK and lateADEp). A reporter gene (mCherry fluorescent protein) is cloned downstream of the minimal core promoter to facilitate the screening process. This reporter gene will be replaced by a therapeutic gene in the SP showing high performance. B) Schematic representation of expected performance. During the screening phase, an expression construct containing the gene for the specific TF is transfected into the cells together with the synthetic promoter. This TF can bind to the responsive elements and induce reporter gene expression in a specific manner.

FIG. 2. General experimental workflow of the screening process using the synthetic promoter library. Synthetic promoters (SPs) were screened in HEK293 by transfection and in Jurkat cell lines by electroporation. During this process, three different constructs are introduced into the cells: the SP, the specific transcription factor (TF), and an EGFP transfection control. After 48 hours, the cells are evaluated by Flow Cytometry Analysis (FACS) quantifying the expression of the reporter gene (mCherry fluorescent protein) and calculating the activation levels (fold induction) by normalization using EGFP expression and control SPs (deficient in TF binding sites). SP candidates are evaluated based on the ability to exhibit superior fold induction with a lower background (minimal reporter gene expression in the absence of transcription factor).

FIG. 3. Activation of synthetic promoters targeting MAF and NR4A2 TF. Synthetic promoters (SPs) were screened in HEK293 and Jurkat cell lines. Fold induction of SP MAF on different minimal promoters used and tested in HEK293 (A) and Jurkat (B) cells, showing different strength. Fold induction of NR4A2 SPs created with the minimal YB_TATA promoter and tested in Jurkat cells (C). Each SP color represents a different TF binding site (TF-BS) used. Fold induction was calculated under normalized conditions using mCherry geometric mean referred to the no-TF condition.

FIG. 4. General structure and functionality of the Lentivirus vector (LV backbone). Synthetic promoters (SPs) showing promising induction potential were cloned into the LV backbone for proof-of-concept testing in T cells. In general, the LV vector is made of two transcriptional units (TUs). The first TU contains 5×TF-BS, the minimal core (mCP), and the sequence encoding the red fluorescent protein, mCherry. The second TU includes a constitutive shEF1a promoter that drives the expression of a transduction-controlling green fluorescent protein, EGFP. These two TUs are separated by an ‘insulating’ sequence (A). HEK293 cells were transduced with these LV constructs, and the EGFP expression was read by flow cytometry and compared to non-transduced cells (B).

FIG. 5. Chronic stimulation scheme for dysfunction model using human CD8+ T cells. To test synthetic promoters (SPs) cloned in LV vectors, an ex vivo dysfunction model was developed. This model was obtained with chronic activation of CD8+ T cells by applying a stimulus with Dynabeads™ (anti-CD3, anti-CD28 antibodies) every two days until day 8 after the first stimulus. Cells were evaluated at days 0, 4, and 8 for markers of exhaustion, functional and cytotoxicity potential. At the end of the protocol, the cells generally resembled exhausted T cells.

FIG. 6. Proof of concept: CD8+ T cells engineered with inducible SPs. Using SPs cloned into LV vectors and the ex vivo model of T cell exhaustion, the inducible SP was used to engineer T cells in a proof-of-concept assay. Cells were isolated and stimulated as described above in the ex vivo Tₑₓ model. At day 4 post-stimulus, cells were transduced with LV containing the SP constructs targeting NR4A2 TF (CON1-NR4A2 BS). Cells were maintained in the Tₑₓ model stimulus regime and evaluated at days 6 and 8 after the first stimulus by FACS. mCherry expression was assessed in EGFP+ cells after gating of dead and double cells using scatter plots (A). The mCherry geometric mean was also evaluated within the EGFP+ population to verify inducible potential (B). The expression of the related TF NR4A2 was measured at day 8 after stimulation by intracellular staining and FACS analysis indicating upregulation during chronic T cell stimulation (C). 1S: Cells stimulated once with 1:1 Dynabeads™ on day 0; 2D: Cells chronically stimulated with 1:1 Dynabeads™ every two days until day 8.

FIG. 7. T cell transduction and ex vivo T cell exhaustion. T CD8+ total cells were isolated from donor buffy-coat and stimulated at day 0 as described above in the workflow (A). Cells were transduced with different lentivirus at day 1 post-stimulus. Non-transduced cells were evaluated regarding the expression of some markers of

T cell exhaustion by immune-staining and FACS analysis. Among the markers, an integral membrane glycoprotein (CD39), two Inhibitory Immune Checkpoint Receptors (PD1, LAG-3) and a TF (NR4A2). Histogram data were analyzed in the CD3+CD8+ population following the gate strategy: Lymphocytes>>Single cells>>Live/Dead>>CD3+CD8+. The Geometric Mean of each marker were extrapolated and plotted in the bar graph.

FIG. 8. Performance of fluorescent reporter during T cell exhaustion. Following the described above ex vivo Tex model (FIG. 7), at day 1 post stimulus, the cells were transduced with LV containing the SP constructs targeting NR4A2 TF and inducing a reporter gene mCherry (A). The cells were kept in the Tex model stimulus regimen and evaluated at days 4, 6 and 8 post first stimulus by FACS. The expression of mCherry was evaluated in EGFP+ cells after gate exclusion of dead and double cells by scatter plots (C). The mCherry geometric mean was also evaluated inside of EGFP+ population to check the inducible potential (B). 1S: resting condition, cells stimulated once with Dynabeads™ 1:1 at day 0; 2D: exhaustion condition, cells stimulated chronically with Dynabeads™ 1:1 every two days until day 8.

FIG. 9. Synthetic Sensors-engineered T CD8+ cells under chronic stimulation induces the release of immunomodulatory molecules to improve T cell tumor killing. The synthetic promoter (SP) targeting NR4A2 transcription factor (TF) which demonstrated promising induction potential in previews experiments with fluorescent reporter were cloned in lentiviral (LV) backbone to guide the expression of immunomodulatory molecules (IL12p70 and CCL21). Using the ex vivo model of T Exhaustion, T cells were engineered as a proof-of-concept test. Cells were isolated and stimulated as described above in the ex vivo Tex model (FIG. 7). At day 1 post stimulus the cells were transduced with LV containing the SP constructs targeting NR4A2 TF (CON1-NR4A2 BSs) and guiding expression of IL12p70 (A) or CCL21 (B). The cells were kept in the Tex model stimulus regimen and evaluated at days 2, 4, 6 and 8 post first stimulus by ELISA evaluating the expression of the cytokine IL-12p70 (A) and chemokine CCL21 (B) in the supernatant collected every two days before the restimulation of the cells. Each day of analysis, the cells were counted, and data is represented as normalized by the cell number in each condition. 1S: Cells stimulated once with Dynabeads™ 1:1 at day 0; 2D: cells stimulated chronically with Dynabeads™ 1:1 every two days until day 8.

### DETAILED DESCRIPTION OF THE INVENTION

The present invention originates from having identified that some engineered nucleic acids have the potential to be sensors of T cell action exhaustion, improving the action and function of CAR T cells, a new reality in the treatment of cancer patients.

The invention therefore concerns a specific engineered nucleic acid construct comprising:
a nucleic acid sequence encoding an inducible synthetic promoter, which is regulated by a protein involved in T cell exhaustion; and
a nucleic acid sequence encoding an immunomodulator under the control of said promoter, said immunomodulator being capable of preventing and/or reversing T cell exhaustion, and of enhancing T cell activation and function.

Promoters or synthetic promoters (SPs) are new engineered DNA sequences that respond to specific transcription factors. Their composite composition enables better control of specificity, orthogonality, and strength of the desired output gene expression. Synthetic promoters that respond to transcription factors differentially expressed in exhausted T cells enable the design of sensors and actuators to reprogram T cell function.

In one embodiment, in the engineered nucleic acid according to the present invention, said promoter comprises at least one binding site for the transcription factor NR4A2 or for the transcription factor MAF.

Mammalian T cells designed with novel, intelligent, inducible synthetic promoters can sense the state of emergency exhaustion and respond to it. The engineered mammalian T cells that sense the exhaustion are reprogrammed to prevent/restore the exhaustion state, thereby enhancing their effector response against the tumor in combination with current CAR T therapy. A couple of strategies were designed in an attempt to counteract T cell dysfunction during CAR T therapy. CAR T cells modified to extrinsically and intrinsically target the PD-1/PD-L1 axis blockade may protect CAR T cells from PD-1 induced exhaustion.

In one embodiment, in the engineered nucleic acid according to the present invention, said immunomodulator is a chemokine or a cytokine that enhances T cell activation and function.

In one embodiment, in the engineered nucleic acid according to the present invention, said immunomodulator is selected from the group consisting of CCL21, CCL19 and IL-12.

The Applicant has surprisingly identified a library of synthetic inducible promoters (SPs) for the transcription factors (TFs) NR4A2 and MAF, which are strongly upregulated during T cell exhaustion.

SPs consist of TF binding sites (TF-BSs) upstream of different minimal core promoters and in different sense-antisense orientations, which drive the expression of a fluorescent reporter. The sensors were screened in HEK293 and Jurkat cell lines to indicate the output induction strength. The different SP configurations allow a wide range of fold induction of the reporter gene, allowing a versatile instrument to be adapted to detect increased levels of the selected TF. The sensors have the potential to differentiate the input levels (e.g. they only detect it when the input is at a high level), allowing for selectivity in the context of exhaustion.

In one embodiment, in fact, in the engineered nucleic acid according to the present invention, said inducible synthetic promoter comprises at least one binding site for the transcription factor NR4A2, wherein,
said binding site of the synthetic promoter is for the transcription factor NR4A2, said binding site has the sequence indicated in SEQ ID NO:6:

Y₁Y₂Y₃Y₄Y₅TY₆TAAAGY₇TCA,

wherein Y₁ to Y₇ are DNA nucleotides selected from the group consisting of A, T, G and C;
wherein if Y₁-Y₅ are present and Y₇ is G, said binding site is for CON1 and has the sequence indicated in SEQ ID NO:8;
wherein if Y₁-Y₅ are present and Y₇ is A, said binding site is for PRD2 and has the sequence indicated in SEQ ID NO:9; and
wherein if Y₁-Y₅ are not present and Y₆ is C, said binding site is for MSH4 and has the sequence indicated in SEQ ID NO:10; or
said binding site has the sequence indicated in SEQ ID NO:7:

AGGTCAY₈Y₉Y₁₀Y₁₁GTGACCT,

wherein Y₈ to Y₁₁ are DNA nucleotides selected from the group consisting of A, T, G and C;
wherein when Y₈ is A, said binding site is for CON2 and has the sequence indicated in SEQ ID NO:11.

In another embodiment, in the engineered nucleic acid according to the present invention, said inducible synthetic promoter comprises at least one binding site for the transcription factor MAF, wherein said binding site has the sequence indicated in SEQ ID NO:1:

AX₁X₂X₃ X₄TGCTGAX₅ X₆ X₇ X₈ X₉ X₁₀ X₁₁ X₁₂,

wherein X₁ to X₁₂ are DNA nucleotides selected from the group consisting of A, T, G and C;
wherein when X₁ is T and X₃ is A, said binding site is for CD69 and has the sequence indicated in SEQ ID NO:2;
wherein when X₁ is T and X₃ is T, said binding site is for SPATA20 and has the sequence indicated in SEQ ID NO:3;
wherein when X₁ is A and X₃ is T, said binding site is for STAT3 and has the sequence indicated in SEQ ID NO:4; and
wherein when X₁ is A and X₃ is A, said binding site is for RNF212 and has the sequence indicated in SEQ ID NO:5.

In one embodiment, in the engineered nucleic acid according to the present invention, said binding sites for the transcription factors (TF-BS) are repeated in the synthetic promoter, preferably said binding sites are repeated in tandem in the range of 2 to 10 tandem repetitions.

The synthetic promoter may be defined as a “sensor device” and has responsive elements or binding sites for transcription factors (TF-BS) cloned in tandem repeats of 2 to 10 tandem repeats, preferably 3 to 5 tandem repeats, separated by spacers, and in combined sense-antisense orientation.

In one embodiment, in the engineered nucleic acid according to the present invention, said promoter comprises at least one spacer, said spacer being a nucleotide sequence in the range of 8 to 25 nucleotides and said spacer being before, between or after the binding sites.

It was surprisingly seen that the TF-BS repeats used in the engineered nucleic acid of the present invention combined with the spacers separating each repeat are unique and provide optimized inducible activity.

In one embodiment, in the engineered nucleic acid according to the present invention, said promoter comprising at least one minimal core promoter, preferably said minimal core is selected from the group consisting of YB_TATA, miniCMV, miniTK and lateADEp.

The transcription factor binding sites (TF-BS) cloned in tandem repeats of 2 to 10 tandem repeats, preferably 3 to 5 tandem repeats, are cloned upstream of the minimal core.

In one embodiment, in the engineered nucleic acid according to the present invention, when said promoter is regulated by the transcription factor MAF, said promoter has a sequence indicated in a sequence selected from the group consisting of SEQ ID NO:12, SEQ ID NO:13, SEQ ID NO:14, SEQ ID NO:15, SEQ ID NO: 16, SEQ ID NO:17, SEQ ID NO:18, SEQ ID NO:19, SEQ ID NO:20, SEQ ID NO: 21, SEQ ID NO:22, SEQ ID NO:23, SEQ ID NO:24, SEQ ID NO:25, SEQ ID NO: 26 or SEQ ID NO:27.

In one embodiment, in the engineered nucleic acid according to the present invention, when said promoter is regulated by the transcription factor NR4A2, said promoter has a sequence indicated in a sequence selected from the group consisting of SEQ ID NO:28, SEQ ID NO:29, SEQ ID NO:30 or SEQ ID NO:31.

It was surprisingly seen that the combination of TF-BS in the promoters according to the sequences SEQ ID NO:12-SEQ ID NO:31 allows the induction of the expression of a fluorescent marker (read-out) or a therapeutic gene.

Furthermore, advantageously, the promoters according to the sequences SEQ ID NO: 12-SEQ ID NO:31 have a short length, of approximately 150 bp, and therefore can be easily engineered and inserted into viral vectors. Native mammalian promoters can have lengths of even multiple kilobases, and therefore be difficult to fit into the limited capacity of a viral vector, even in lentiviruses.

In a second aspect, the present invention concerns a virus comprising the engineered nucleic acid.

Furthermore, as a proof of concept, we engineered T cells with the object of producing cells capable of sensing the exhaustion signal (TF) and responding to it by producing a reporter/actuator molecule.

Compact synthetic promoters are alternatives to endogenous ones and may show greater specificity for a human cellular state. The synthetic nucleic acid promoters according to the invention are regulated by the same endogenous transcription factors associated with the native promoters and are designed to drive the expression of a reporter or therapeutic protein. Furthermore, these SPs can be designed to allow control at the transcription level, allowing fine-tuning of the function thereof.

In a third aspect, the present invention concerns a cell comprising the engineered nucleic acid.

Surprisingly, T cells engineered with these smart nucleic acid devices can sense T cell exhaustion by sensing transcription factors (TFs) and respond to it through controlled expression of a transcribed “output” gene (see FIG. 6).

The different TF-BSs in combination with the minimal cores allow the construction of a library made of SPs with a wide range of activation potential strength, which is extremely important in a device to be applied in cell therapy.

The invention described herein relates to new transcriptional sensors of T cell exhaustion that can be exploited in immunotherapy against cancer and other inflammatory diseases. Without being tied to any theory, it can be asserted that the sensor with its distinctive characteristics is specific, exclusive, modulable, and can be applied to engineered T cells to improve CAR T therapy on exhausting cells.

In a fourth aspect, the cell comprising the engineered nucleic acid according to the present invention or the virus comprising the engineered nucleic acid according to the present invention, for use as a medicinal product, is described.

In a fifth aspect, the cell comprising the engineered nucleic acid according to the present invention or the virus comprising the engineered nucleic acid according to the present invention, for use in the treatment of cancer, is described.

In a sixth aspect, the cell comprising the engineered nucleic acid according to the present invention or the virus comprising the engineered nucleic acid according to the present invention, for use in immunotherapy, is described.

In a seventh aspect, the cell comprising the engineered nucleic acid according to the present invention or the virus comprising the engineered nucleic acid according to the present invention, for inducing the activity of a chimeric antigen receptor (CAR) T cell is described.

Surprisingly, in fact, the engineered nucleic acid or “sensor”, with its distinctive characteristics, is specific, exclusive, modulable, and can be applied to engineered T cells to improve CAR T therapy on cells undergoing exhaustion.

In an eighth aspect, the cell comprising the engineered nucleic acid according to the present invention or the virus comprising the engineered nucleic acid according to the present invention, for sensing the exhaustion/dysfunctional state of a chimeric antigen receptor (CAR) T cell, is described.

In a ninth aspect, the cell comprising the engineered nucleic acid according to the present invention or the virus comprising the engineered nucleic acid according to the present invention, for regulating gene expression in cell therapy by a chimeric antigen receptor (CAR) T cell, is described.

In a further aspect, a treatment method using the engineered nucleic acid according to the present invention, the cell comprising the engineered nucleic acid according to the present invention or the virus comprising the engineered nucleic acid according to the present invention is described.

In the present invention when the following definition is used:
“MAF” is intended to comprise the transcription factor Maf, also known as proto-oncogene c-Maf or V-maf, the homolog of the musculoaponeurotic fibrosarcoma oncogene, a transcription factor that in humans is encoded by the MAF gene (UniProt: O75444);
“NR4A2” is intended to mean the intracellular transcription factor, a member of the nuclear receptor family, which in humans is encoded by the NR4A2 gene (UniProt: P43354).
“CCL21” and “CCL19” belong to a large family of small secreted proteins that signal through cell surface G protein-coupled heptahelical chemokine receptors on the cell surface. In humans they are encoded by the CCL21 and CCL19 genes. They are genes located on the short arm of chromosome 9, which encode the CC cytokine cluster.
“IL-12” is intended to mean interleukin 12. An interleukin is a type of cytokine that regulates immune responses. Interleukin-12 (IL-12) is a covalently linked heterodimer made of a light chain of about 35 kDa (p35 or IL-12a) and a heavy chain of about 40 kDa (p40 or IL-12B). The two genes encoding p40 and p35 are unrelated and located on separate chromosomes (5q31-33 and 3p12-q13.2, respectively, in humans).

The engineered nucleic acids according to the invention are extremely versatile sensors in application. They can be used coupled to any output gene of interest to obtain any desired sensor-actuator device. They can be used for the cell type of interest, wherein the transcription factor upregulation is associated with a specific cellular state. Cells can be designed with sensor devices and function for drug screening approaches, wherein the transcription factor is the drug target or a cellular state marker or it is involved in the targeted pathway. Some of the possible fields of application are autoimmunity, immuno-oncology, Parkinson sensing and rheumatoid arthritis, wherein these sensors can recognize transcription factors involved in these pathways.

SEQ ID NO:1: AX₁X₂X₃ X₄TGCTGAX₅X₆X₇X₈X₉X₁₀X₁₁X₁₂, wherein X₁ to X₁₂ are DNA nucleotides selected from the group consisting of A, T, G and C.

SEQ ID NO:6: Y₁Y₂Y₃Y₄YSTY₆TAAAG Y₇ TCA, wherein Y₁ to Y₇ are DNA nucleotides selected from the group consisting of A, T, G and C.

SEQ ID NO:7: AGGTCAY₈Y₉Y₁₀Y₁₁GTGACCT, wherein Y₈ to Y₁₁ are DNA nucleotides selected from the group consisting of A, T, G and C.

MAF targeting SPs
Bold = TF-BSs, Unbold = spacers, Underlined = minimal core promoter
SP33 (5xCD69 BSs-YB_TATA)_SEQ ID NO: 12
GATCTTTGTTTTGATTAAATAATTGCTGATGTAATGTCGCGATGTACGTAAGA
TAATTGCTGATGTAATGTTGCTGATAAGATAATTGCTGATGTAATGTAGACGT
GGCAGTTAGATAATTGCTGATGTAATGTGGGGATTAAGATAATTGCTGATGT
AATGTGCGATTAAGCTTTCCAATATGCTCTAGAGGGTATATAATGGGGGCCA
SP34 (5xCD69 BSs-miniCMV)_SEQ ID NO: 13
GATCTTTGTTTTGATTAAATAATTGCTGATGTAATGTCGCGATGTACGTAAGA
TAATTGCTGATGTAATGTTGCTGATAAGATAATTGCTGATGTAATGTAGACGT
GGCAGTAAGATAATTGCTGATGTAATGTGGGGATTAAGATAATTGCTGATGT
AATGTGCGATTAAGCTTTCCAATATGCGTAGGCGTGTACGGTGGGAGGTCTA
TATAAGCAGAGCTCGTTTAGTGAACCGTCAGATC
SP35 (5xCD69 BSs-miniTK)_SEQ ID NO: 14
GATCTTTGTTTTGATTAAATAATTGCTGATGTAATGTCGCGATGTACGTAAGA
TAATTGCTGATGTAATGTTGCTGATAAGATAATTGCTGATGTAATGTAGACGT
GGCAGTAAGATAATTGCTGATGTAATGTGGGGATTAAGATAATTGCTGATGT
AATGTGCGATTAAGCTTTCCAATATGCTTCGCATATTAAGGTGACGCGTGTGG
CCTCGAACACCGAGCGACCCTGCAGCGACCCGCTTAA
SP36 (5xCD69 BSs-lateADEp)_SEQ ID NO: 15
GATCTTTGTTTTGATTAAATAATTGCTGATGTAATGTCGCGATGTACGTAAGA
TAATTGCTGATGTAATGTTGCTGATAAGATAATTGCTGATGTAATGTAGACGT
GGCAGTAAGATAATTGCTGATGTAATGTGGGGATTAAGATAATTGCTGATGT
AATGTGCGATTAAGCTTTCCAATATGCAGACGCTAGCGGGGGGCTATAAAAG
GGGGTGGGGGCGTTCGTCCTCACTCT
SP37 (5xSPATA20 BSs-YB_TATA)_SEQ ID NO: 16
GATCTTTGTTTTGATTAAATATTTGCTGAATTAAATCCGCGATGTACGTAAGA
TATTTGCTGAATTAAATCTGCTGATAAGATATTTGCTGAATTAAATCAGACGT
GGCAGTTAGATATTTGCTGAATTAAATCGGGGATTAAGATATTTGCTGAATTA
AATCGCGATTAAGCTTTCCAATATGCTCTAGAGGGTATATAATGGGGGCCA
SP38 (5xSPATA20 BSs-miniCMV)_SEQ ID NO: 17
GATCTTTGTTTTGATTAAATATTTGCTGAATTAAATCCGCGATGTACGTAAGA
TATTTGCTGAATTAAATCTGCTGATAAGATATTTGCTGAATTAAATCAGACGT
GGCAGTAAGATATTTGCTGAATTAAATCGGGGATTAAGATATTTGCTGAATT
AAATCGCGATTAAGCTTTCCAATATGCGTAGGCGTGTACGGTGGGAGGTCTA
TATAAGCAGAGCTCGTTTAGTGAACCGTCAGATC
SP39 (5xSPATA20 BSs-miniTK)_SEQ ID NO: 18
GATCTTTGTTTTGATTAAATATTTGCTGAATTAAATCCGCGATGTACGTAAGA
TATTTGCTGAATTAAATCTGCTGATAAGATATTTGCTGAATTAAATCAGACGT
GGCAGTAAGATATTTGCTGAATTAAATCGGGGATTAAGATATTTGCTGAATT
AAATCGCGATTAAGCTTTCCAATATGCTTCGCATATTAAGGTGACGCGTGTGG
CCTCGAACACCGAGCGACCCTGCAGCGACCCGCTTAA
SP40 (5xSPATA20 BSs-lateADEp)_SEQ ID NO: 19
GATCTTTGTTTTGATTAAATATTTGCTGAATTAAATCCGCGATGTACGTAAGA
TATTTGCTGAATTAAATCTGCTGATAAGATATTTGCTGAATTAAATCAGACGT
GGCAGTAAGATATTTGCTGAATTAAATCGGGGATTAAGATATTTGCTGAATT
AAATCGCGATTAAGCTTTCCAATATGCAGACGCTAGCGGGGGGCTATAAAAG
GGGGTGGGGGCGTTCGTCCTCACTCT
SP41 (5xSTAT3 BSs-YB_TATA)_SEQ ID NO: 20
GATCTTTGTTTTGATTAAAAATGTGCTGACTCAGAGACGCGATGTACGTAAG
AAATGTGCTGACTCAGAGATGCTGATAAGAAATGTGCTGACTCAGAGAAGA
CGTGGCAGTTAGAAATGTGCTGACTCAGAGAGGGGATTAAGAAATGTGCTG
ACTCAGAGAGCGATTAAGCTTTCCAATATGCTCTAGAGGGTATATAATGGGG
GCCA
SP42 (5xSTAT3 BSs-miniCMV)_SEQ ID NO: 21
GATCTTTGTTTTGATTAAAAATGTGCTGACTCAGAGACGCGATGTACGTAAG
AAATGTGCTGACTCAGAGATGCTGATAAGAAATGTGCTGACTCAGAGAAGA
CGTGGCAGTAAGAAATGTGCTGACTCAGAGAGGGGATTAAGAAATGTGCTG
ACTCAGAGAGCGATTAAGCTTTCCAATATGCGTAGGCGTGTACGGTGGGAG
GTCTATATAAGCAGAGCTCGTTTAGTGAACCGTCAGATC
SP43 (5xSTAT3 BSs-miniTK)_SEQ ID NO: 22
GATCTTTGTTTTGATTAAAAATGTGCTGACTCAGAGACGCGATGTACGTAAG
AAATGTGCTGACTCAGAGATGCTGATAAGAAATGTGCTGACTCAGAGAAGA
CGTGGCAGTAAGAAATGTGCTGACTCAGAGAGGGGATTAAGAAATGTGCTG
ACTCAGAGAGCGATTAAGCTTTCCAATATGCTTCGCATATTAAGGTGACGCG
TGTGGCCTCGAACACCGAGCGACCCTGCAGCGACCCGCTTAA
SP44 (5xSTAT3 BSs-lateADEp)_SEQ ID NO: 23
GATCTTTGTTTTGATTAAAAATGTGCTGACTCAGAGACGCGATGTACGTAAG
AAATGTGCTGACTCAGAGATGCTGATAAGAAATGTGCTGACTCAGAGAAGA
CGTGGCAGTTAGAAATGTGCTGACTCAGAGAGGGGATTAAGAAATGTGCTG
ACTCAGAGAGCGATTAAGCTTTCCAATATGCAGACGCTAGCGGGGGGCTATA
AAAGGGGGTGGGGGCGTTCGTCCTCACTCT
SP45 (5xRNF212 BSs-YB_TATA)_SEQ ID NO: 24
GATCTTTGTTTTGATTAAAATAGTGCTGATGCTGTGTCGCGATGTACGTAAGA
ATAGTGCTGATGCTGTGTTGCTGATAAGAATAGTGCTGATGCTGTGTAGACG
TGGCAGTAAGAATAGTGCTGATGCTGTGTGGGGATTAAGAATAGTGCTGATG
CTGTGTGCGATTAAGCTTCCAATATGCTCTAGAGGGTATATAATGGGGGCCA
SP46 (5xRNF212 BSs-miniCMV)_SEQ ID NO: 25
GATCTTTGTTTTGATTAAAATAGTGCTGATGCTGTGTCGCGATGTACGTAAGA
ATAGTGCTGATGCTGTGTTGCTGATAAGAATAGTGCTGATGCTGTGTAGACG
TGGCAGTAAGAATAGTGCTGATGCTGTGTGGGGATTAAGAATAGTGCTGATG
CTGTGTGCGATTAAGCTTTCCAATATGCGTAGGCGTGTACGGTGGGAGGTCT
ATATAAGCAGAGCTCGTTTAGTGAACCGTCAGATC
SP47 (5xRNF212 BSs-miniTK)_SEQ ID NO: 26
GATCTTTGTTTTGATTAAAATAGTGCTGATGCTGTGTCGCGATGTACGTAAGA
ATAGTGCTGATGCTGTGTTGCTGATAAGAATAGTGCTGATGCTGTGTAGACG
TGGCAGTAAGAATAGTGCTGATGCTGTGTGGGGATTAAGAATAGTGCTGATG
CTGTGTGCGATTAAGCTTTCCAATATGCTTCGCATATTAAGGTGACGCGTGTG
GCCTCGAACACCGAGCGACCCTGCAGCGACCCGCTTAA
SP48 (5xRNF212 BSs-lateADEp)_SEQ ID NO: 27
GATCTTTGTTTTGATTAAAATAGTGCTGATGCTGTGTCGCGATGTACGTAAGA
ATAGTGCTGATGCTGTGTTGCTGATAAGAATAGTGCTGATGCTGTGTAGACG
TGGCAGTAAGAATAGTGCTGATGCTGTGTGGGGATTAAGAATAGTGCTGATG
CTGTGTGCGATTAAGCTTTCCAATATGCAGACGCTAGCGGGGGGCTATAAAA
GGGGGTGGGGGCGTTCGTCCTCACTCT
NR4A2 targeting SPs
Bold = TF-BSs, Unbold = spacers, Underlined = minimal core promoter
SP73 (5xCON1 BSs-YB_TATA)_SEQ ID NO: 28
GATCTTTGTTTTGATTAATGACCTTTAAAGGTCACGCGATGTACGTAAGTGAC
CTTTAAAGGTCATGCTGATAAGTGACCTTTAAAGGTCAAGACGTGGCAGTAA
GTGACCTTTAAAGGTCAGGGGATTAAGTGACCTTTAAAGGTCAGCGATTAAG
CTTTCCAATATGCTCTAGAGGGTATATAATGGGGGCCA
SP74 (5xCON2 BSs-YB_TATA)_SEQ ID NO: 29
GATCTTTGTTTTGATTAAAGGTCAACTTGTGACCTCGCGATGTACGTAAGAGG
TCAACTTGTGACCTTGCTGATAAGAGGTCAACTTGTGACCTAGACGTGGCAG
TAAGAGGTCAACTTGTGACCTGGGGATTAAGAGGTCAACTTGTGACCTGCG
ATTAAGCTTTCCAATATGCTCTAGAGGGTATATAATGGGGGCCA
SP75 (5xMSH4 BSs-YB_TATA)_SEQ ID NO: 30
GATCTTTGTTTTGATTAATCTAAAGGTCACGCGATGTACGTAAGTCTAAAGGT
CATGCTGATAAGTCTAAAGGTCAAGACGTGGCAGTAAGTCTAAAGGTCAGG
GGATTAAGTCTAAAGGTCAGCGATTAAGCTTTCCAATATGCTCTAGAGGGTAT
ATAATGGGGGCCA
SP78 (5xPRD2 BSs-YB_TATA)_SEQ ID NO: 31
GATCTTTGTTTTGATTAATGACCTTTAAAGATCACGCGATGTACGTAAGTGAC
CTTTAAAGATCATGCTGATAAGTGACCTTTAAAGATCAAGACGTGGCAGTAA
GTGACCTTTAAAGATCAGGGGATTAAGTGACCTTTAAAGATCAGCGATTAAG
CTTTCCAATATGCTCTAGAGGGTATATAATGGGGGCCA

Below are Examples of embodiments of the present invention provided for illustrative purposes. The present invention is described with reference to the following examples.

### EXAMPLES

### Example 1: Design of Engineered Nucleic Acids

The invention comprises novel engineered nucleic acids, which are transcriptional sensors of T cell exhaustion. These sensors are synthetic promoters designed to detect specific transcription factors upregulated in exhausted T cells. Synthetic promoters are genetic structures made of a regulatory region, i.e. the specific binding site of the transcription factor, and a minimal core promoter to start the transcription of a reporter gene.

We started from a list of TFs upregulated in exhausted T cells, and MAF and NR4A2 were selected, whose consensus TF binding sites (TF-BS) and variants thereof were selected.

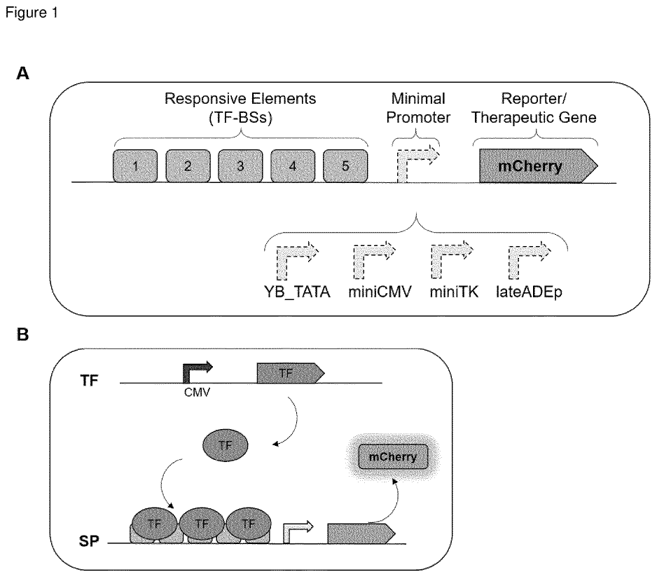

These transcription factor binding sites (TF-BS) were designed to be cloned as a repeat, for example in tandem 5× (5 repeats) upstream of a minimal core promoter, with specific spacers between each repeat (each spacer, for example, has a length of 10-15 nucleotides (bps)) and from the last binding site to the minimal core promoter (a length, for example, of 20 bps). Several minimal core promoters (YB-TATA, miniCMV, miniTK, lateADEp) were tested in combination with TF-BS to drive the expression of the fluorescent protein mCherry (FIG. 1A). The entire promoter library (SP) according to the invention, having the sequences SEQ ID NO:12-SEQ ID NO: 31 was tested in a screening process to find the best candidates.

The expected scenario during this screening phase is the induction of mCherry fluorescence following the expression of the transcription factor TF (FIG. 1B).

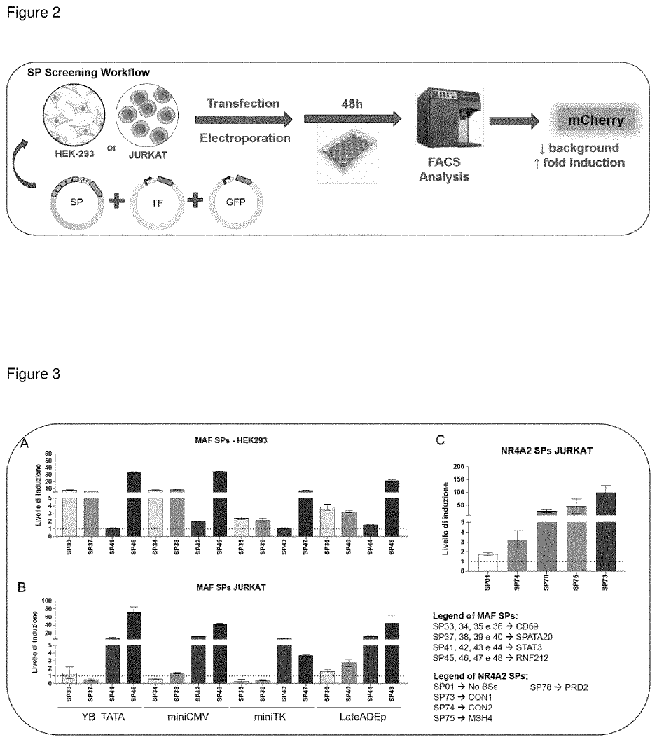

The generated promoter library was screened in HEK293 and Jurkat cell lines by co-transfection/electroporation of plasmids for SP, +/− the plasmid expressing the cognate transcription factor, and a transfection control plasmid expressing EGFP. Cells were analysed by flow cytometry 48 hours after transfection, wherein the geometric mean fluorescence intensity of mCherry and EGFP were quantified, normalized, and expressed as fold induction (FIG. 2).

Using the no-TF condition as a reference for each specific promoter, the performance of candidate promoters is measured based on their ability to exhibit high fold induction with low background in the OFF state.

The transcription factor binding sites (TF-BSs) of MAF and NR4A2 are listed in Table 1. The MAF-specific engineered nucleic acids exhibit a wide range of fold induction that is essential while choosing to drive the expression of a therapeutic molecule (FIG. 3A). When specific engineered nucleic acids were tested in Jurkat cells, they were also inducible but some of them had a different strength when compared to that in HEK293 cells, demonstrating that they depend on the cellular context (FIG. 3B). Among the minimal promoters tested, YB_TATA showed one of the most consistent fold inductions and was selected to drive the NR4A2 promoter constructs. Of particular interest, engineered nucleic acids YB_TATA NR4A2 showed the highest fold induction in Jurkat cells compared to SP MAFs, with a wide range of induction compared to the non-binding site (BS) control SP01 (FIG. 3C).

TABLE 1
Transcription factor binding sites used for
the promoters MAF and NR4A2

| TF | BS | | SEQ ID |
|---|---|---|---|
| Target | Names | BS Sequences | NO |
| NR4A2 | CON1ª | TGACCTTTAAAGGTCA | 8 |
| | PRD2 | TGACCTTTAAAGATCA | 9 |
| | MSH4 | _____TCTAAAGGTCA | 10 |
| | CON2ª | AGGTCAACTTGTGACCT | 11 |
| MAF | CD69ª | ATAATTGCTGATGTAATGT | 2 |
| | SPATA20 | ATATTTGCTGAATTAAATC | 3 |
| | STAT3 | AAATGTGCTGACTCAGAGA | 4 |
| | RNF212 | AATAGTGCTGATGCTGTGT | 5 |
| | CON1ª | TGACCTTTAAAGGTCA | 8 |
| | PRD2 | TGACCTTTAAAGATCA | 9 |

^{a}consensus sequence.
Bold = differences between consensus and variants.

Overall, the nucleic acids engineered and designed according to the present invention, targeting MAF and NR4A2, are inducible and exhibit a broad range of inducible activities that allows us to more finely control the expression of a therapeutic gene under its control.

### Cloning in Lentivirus Vectors

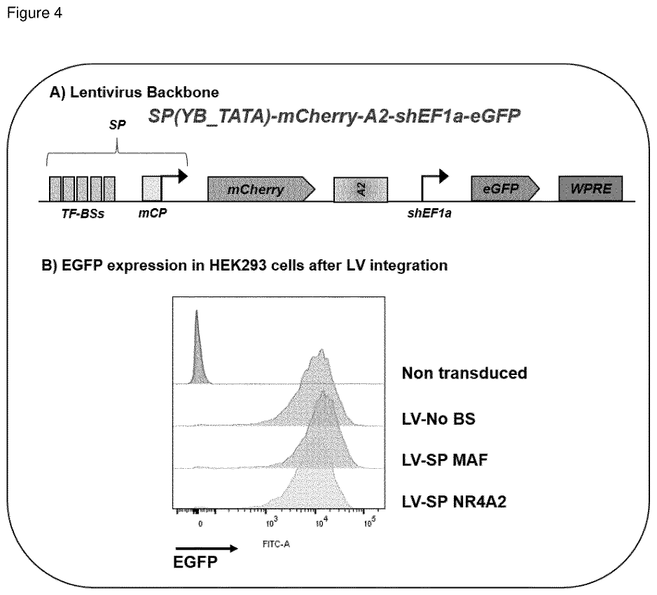

Considering that our ultimate goal is to engineer T cells by infecting them with lentiviruses harbouring our constructs, the synthetic inducible promoters were then cloned into lentivirus vectors using the Golden Gate cloning system and could then be tested in T cells as a proof of concept. The overall structure of the promoter in the lentivirus backbone is shown in FIG. 4 and is made of a first transcription unit (TU) containing an example of the original SP structure (5×TF-BS-YB_TATA-mCherry) and a second TU containing a constitutive promoter inducing EGFP expression as a transduction control (shEF1a-EGFP) isolated from insulator A2. The LV constructs (nucleic acid engineered according to the invention) were transduced into HEK293 cells and EGFP expression was quantified via FACS, demonstrating that the constructs were successfully integrated (FIG. 4B). Results regarding the functionality of these lentivirus constructs in T cells are described in Example 2.

### Example 2: Exhaustion Model Using Ex-Vivo CD8+ T Cells

The synthetic inducible promoters of the present invention have shown promising potential as sensors of transcription factors markers of T cell exhaustion, and an ex vivo model to simulate T cell exhaustion was developed by our group.

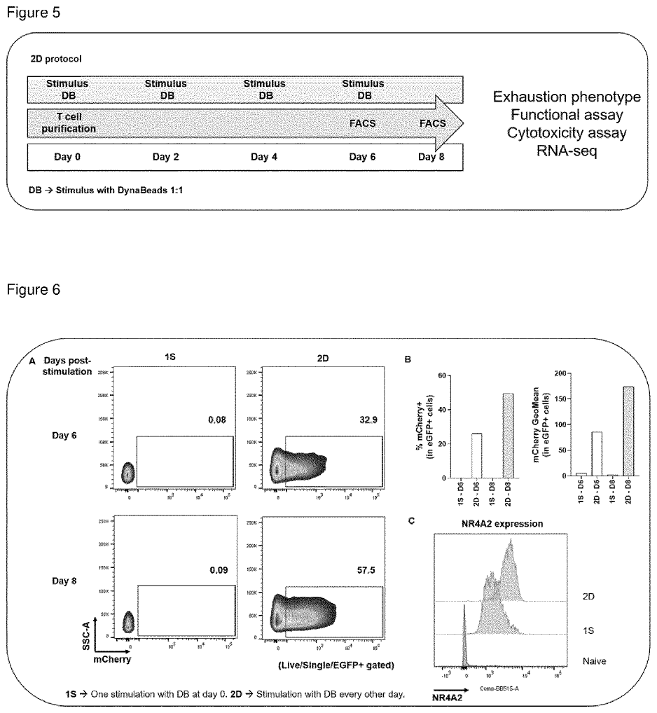

In this model, human CD8+ T cells were purified from buffy-coats of healthy donors by negative selection. These cells were chronically stimulated with Dynabeads™ 1:1 (beads: cells), every other day until day 8 after the first stimulus. At days 0, 4, and 8, the cells were analysed by immunostaining and flow cytometry (FACS) querying exhaustion markers as well as functional and cytotoxicity potential. We observed that, at day 8, the cells showed general characteristics of exhaustion, with upregulation of TFs such as NR4A2 (FIG. 6C). As a control we used cells stimulated only once as for T cell activation (FIG. 5).

### Example 3: Proof of Concept: Engineering CD8+ T Cells with Inducible Synthetic Promoters that Sense Transcription Factors Specific to T Exhaustion

This invention was tested to engineer ex vivo human CD8+ T cells using the protocol described above. CD8+ T cells were purified from healthy human buffy-coats, stimulated, and transduced with Lentivirus particles containing the synthetic promoter sensor targeting the transcription factor NR4A2 (CON1-NR4A2 BSs). As part of an exhaustion protocol, in which cells were stimulated for 8 days, sensor activation was assessed by FACS at days 6 and 8. We observed from the expression of a reporter gene (mCherry) that our SP was able to sense exhaustion at day 6 and increased the percentage of positive cells by day 8 (FIG. 6A). Furthermore, an increase in fluorescence intensity (mCherry Geometric mean) over time was also noted, meaning that SPs are inducible and can potentially be stimulated during exhaustion development (FIG. 6B). As expected, the corresponding TF NR4A2 increases after Dynabeads (1S) stimulation and accumulates with chronic stimulation at day 8 after stimulation, thus demonstrating its association with the exhaustion phenotype, as already reported in the literature (2D, FIG. 6C).

### Example 4: Engineering T and CAR T Cells with Synthetic Promoters in a Tumor Model, Evaluating Regression and Preventing Exhaustion

CCL21 and CCL19 (chemokines that promote trafficking of T cells to tumor sites) and IL-12 (a cytokine that enhances T cell activation and function) were shown to be immunomodulators that can reinvigorate T cells. Synergistic antitumor activity between transferred and endogenous cells, and an increase in overall therapeutic efficacy in solid and liquid tumor models were demonstrated.

Another approach is to engineer T cells that can sense markers of exhaustion and act by producing an inactive form of Granzyme B (SUMO-Granzyme B) which is active only in tumor cells and can induce cell apoptosis through caspase pathways.

### Example 5: The NR4A2 SP (CON1) Guides the Expression of IL-12p70 and CCL21 Immunomodulators in T CD8+ Engineered Cells Under Exhaustion Stimulus

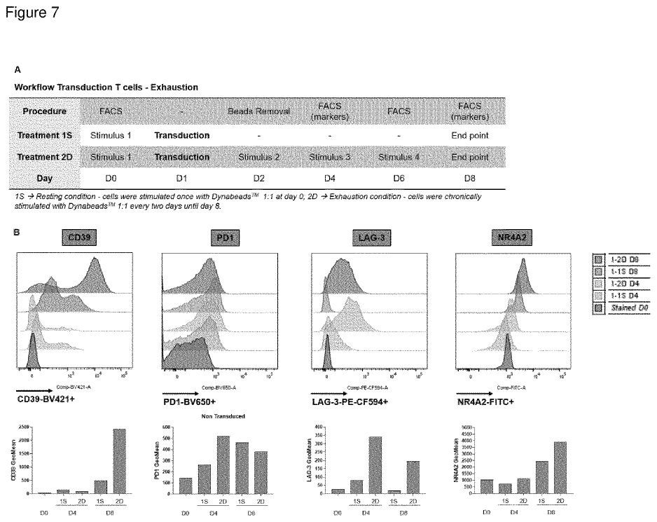

The potential of the SPs proposed here as sensor for T cell exhaustion were tested in our ex vivo T cell exhaustion model to drive an immunomodulatory molecule production. This will serve as a proof-of-concept that it can be used to improve the anti-cancer therapies. Briefly, in this model, human total T CD8+ cells were purified from health donor buffy-coats by negative selection. These cells were chronically stimulated with Dynabeads™ 1:1 (beads: cells) every other day until day 8 post first stimulus. At days 0, 4 and 8 the cells were analyzed by immuno-staining and Flow Cytometry (FACS) questioning the markers of exhaustion as well as functionality of fluorescent reporter or immunomodulatory molecule release (FIG. 7A). During the model characterization, it was observed that at day 8 the cells chronically stimulated (2D) showed overall characteristics of exhaustion with overexpression of TF NR4A2 and membrane glycoprotein CD39 (FIG. 7B). Instead, other markers like the Inhibitory Immune Checkpoint Receptors PD1 and LAG-3 showed a higher expression on day 4 under chronic stimulation, indicating that they have a different dynamic of expression to induce exhaustion in T cells (FIG. 7B).

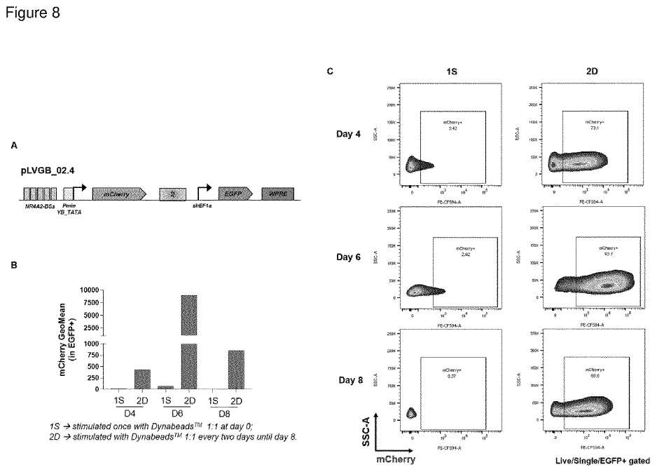

The T cells in this model were transduced at day 1 post first stimulus with Lentivirus particles containing the synthetic promoter sensor targeting NR4A2 TF (LVGB_02.4, FIG. 8A), which induce the expression of a reporter fluorescent protein mCherry. Under a protocol of exhaustion where the cells were stimulated for 8 days, activation of the sensor was evaluated by FACS at days 4, 6 and 8. It was observed, by the expression of a fluorescent transduction marker EGFP, that approx. 45% transduction efficiency at day 4 post first stimulus were obtained. Upon stimulation, it was also observed by the expression of mCherry that the SP sensor responding to NR4A2 TF increased over the days of stimulation, confirming that it can effectively sense the upregulation of the exhaustion-associated TF (FIG. 8C, 1S cells stimulated once, 2D, cells chronically stimulated). Moreover, the increase was noticed also in the fluorescence intensity (mCherry Geomean) over the time, meaning that the SPs are inducible and can potentially be stimulated during exhaustion development (FIG. 8B). These data are in line with the increased levels of NR4A2 after dynabeads stimulation (1S) and its accumulation in the chronic stimulation at day 8 post stimulation (FIG. 7B).

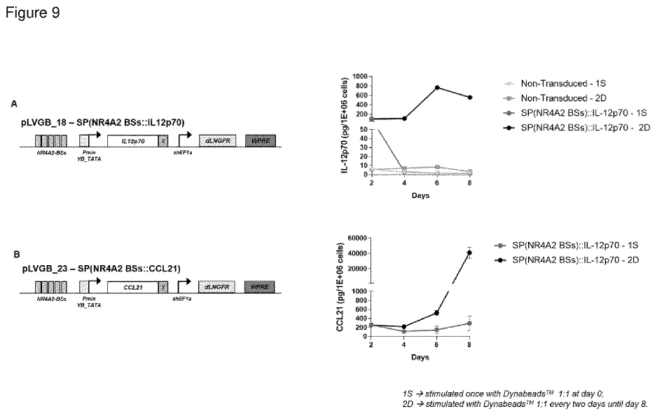

The synthetic promoter targeting NR4A2 TF was cloned in the LV backbone in a new configuration in which the reporter gene is an immunostimulatory molecule IL-12p70 or CCL21 to show the potential of this devices to sense exhaustion and respond by releasing an immunomodulatory molecule. In this new LV configuration, also the control of transduction guided by a constitutive promoter was replaced to a non-fluorescent membrane-bound truncated receptor LNGFR, which can be tracked by extracellular immunostaining. The T cells were transduced at day 1 post first stimulus with lentiviral particles containing the synthetic promoter sensor targeting NR4A2 TF and inducing the expression of an actuator molecule IL12p70 (pLVGB_18) or CCL21 (pLVGB_23). Under a protocol of exhaustion where the cells were stimulated for 8 days, activation of the sensor was evaluated on the supernatant of the cells by ELISA at days 2, 4, 6 and 8. It was observed, by the expression and staining of a membrane transduction maker (LNGFR), that a transduction efficiency ranging from 24 to 39% at day 6 post first stimulus was obtained. The ELISA quantification demonstrated that the SP sensor targeting NR4A2 TF was able to sense the exhaustion marker and produce IL12p70 (FIG. 9A) and CCL21 (FIG. 9B) since day 2 of the protocol, with an increase in the release across the chronic stimulation. Conversely, in resting condition (1S) the high level of IL12p70 is detected only at day 2 and then goes down across the days, indicating a decreased level of activation of our SP sensor. Moreover, CCL21 expression is also maintained constant and low in resting condition (1S), indicating that the sensor shows very low activity (FIGS. 9A and B, 1S). These data corroborate with the data shown before on the fluorescent reporter and with the level of the TF NR4A2 after dynabeads stimulation.

Altogether, the Synthetic Sensors-engineered T CD8+ cells under chronic stimulation induces the release of therapeutic immunomodulatory molecules, which can improve the immune system in the tumor microenvironment, improving the T cell tumor killing.

From the detailed description and the Examples reported above, the advantages achieved by the engineered nucleic acid of the present invention as a sensor of T cell exhaustion are apparent. In particular, these sensors were shown to be surprisingly and advantageously suitable for sensing exhaustion/dysfunctional state of a chimeric antigen receptor (CAR) T cell.

1-18. (canceled)

19. An engineered nucleic acid comprising:
a nucleic acid sequence encoding an inducible synthetic promoter, which is regulated by a protein involved in T cell exhaustion; and
a nucleic acid sequence encoding an immunomodulator under the control of said promoter, said immunomodulator being capable of preventing and/or reversing T cell exhaustion, and of enhancing T cell activation and function.

20. The engineered nucleic acid according to claim 19, wherein said promoter comprises at least one binding site for the transcription factor NR4A2 or transcription factor MAF.

21. The engineered nucleic acid according to claim 19, wherein said immunomodulator is selected from the group consisting of CCL21, CCL19 and IL-12.

22. The engineered nucleic acid according to claim 21, wherein said binding sites for transcription factors (TF-BS) are repeated in the synthetic promoter, preferably said binding sites are repeated in tandem in the range of 2 to 10 tandem repetitions.

23. The engineered nucleic acid according to claim 19, wherein said promoter comprises at least one spacer, said spacer being a nucleotide sequence in the range of 8 to 25 nucleotides and said spacer being before, between or after the binding sites.

24. The engineered nucleic acid according to claim 19, said promoter comprising at least one minimal core promoter, preferably said minimal core promoter is selected from the group consisting of YB_TATA, miniCMV, miniTK and lateADEp.

25. The engineered nucleic acid according to claim 19, wherein said inducible synthetic promoter comprises at least one binding site for the transcription factor wherein,
said binding site has the sequence indicated in SEQ ID NO:6:
Y₁Y₂Y₃Y₄Y₅TY₆TAAAGY₇TCA,
wherein Y₁ to Y₇ are DNA nucleotides selected from the group consisting of A, T, G and C;
wherein if Y₁-Y₅ are present and Y₇ is G, said binding site is for CON1 and has the sequence indicated in SEQ ID NO:8;
wherein if Y₁-Y₅ are present and Y₇ is A, said binding site is for PRD2 and has the sequence indicated in SEQ ID NO:9; and
wherein if Y₁-Y₅ are not present and Y₆ is C, said binding site is for MSH4 and has the sequence indicated in SEQ ID NO:10; or
said binding site has the sequence indicated in SEQ ID NO:7:
AGGTCAY₈Y₉Y₁₀Y₁₁GTGACCT,
wherein Y₈ to Y₁₁ are DNA nucleotides selected from the group consisting of A, T, G and C;
wherein when Y₈ is A, said binding site is for CON2 and has the sequence indicated in SEQ ID NO: 11.

26. The engineered nucleic acid according to claim 25, wherein when said promoter is regulated by the transcription factor NR4A2, said promoter has a sequence indicated in a sequence selected from the group consisting of SEQ ID NO:28 to SEQ ID NO:31.

27. The engineered nucleic acid according to claim 19, wherein said inducible synthetic promoter comprises at least one binding site for the transcription factor MAF, wherein said binding site has the sequence indicated in SEQ ID NO:1:
AX₁X₂X₃ X₄TGCTGAX₅ X₆ X₇ X₈ X₉ X₁₀ X₁₁ X₁₂,
wherein X₁ to X₁₂ are DNA nucleotides selected from the group consisting of A, T, G and C.

28. The engineered nucleic acid according to claim 27, wherein when said promoter is regulated by the transcription factor MAF, said promoter has a sequence selected from the group consisting of SEQ ID NO: 12-SEQ ID NO:27.

29. A method for treating a cancer in a subject in need thereof, said method comprising administering an engineered nucleic acid according to claim 19.

30. A method for immunotherapy in a subject in need thereof, said method comprising administering an engineered nucleic acid according to claim 19.

31. The method according to claim 29, wherein said engineered nucleic acid induces the activity of a chimeric antigen receptor (CAR) T cell.

32. The method according to claim 29, wherein said engineered nucleic acid senses the exhaustion/state of dysfunction of a chimeric antigen receptor (CAR) T cell.

33. The method according to claim 29, wherein said engineered nucleic acid regulates gene expression in cell therapy by a chimeric antigen receptor (CAR) T cell.

34. The method according to claim 30, wherein said engineered nucleic acid induces the activity of a chimeric antigen receptor (CAR) T cell.

35. The method according to claim 30, wherein said engineered nucleic acid senses the exhaustion/state of dysfunction of a chimeric antigen receptor (CAR) T cell.

36. The method according to claim 30, wherein said engineered nucleic acid regulates gene expression in cell therapy by a chimeric antigen receptor (CAR) T cell.
